(19) United States
(12) Patent Application Publication
(10) Pub. No.: US 2018/0221188 A1
(43) Pub. Date: Aug. 9, 2018

(54) ORTHOTIC APPLIANCE WITH CONTINUOUSLY ADJUSTABLE POSITIONING OF CORRECTIVE ELEMENTS

(71) Applicant: Motion Therapeutics, Inc., San Francisco, CA (US)

(72) Inventors: CYNTHIA GIBSON-HORN, Oakland, CA (US); David Pearson, Thousand Oaks, CA (US); Mark Sunderland, Philadelphia, PA (US); Andrew Lesher, Wynnewood, PA (US); Elle Abbey, Philadelphia, PA (US)

(73) Assignee: Motion Therapeutics, Inc., San Francisco, CA (US)

(21) Appl. No.: 15/425,602

(22) Filed: Feb. 6, 2017

(51) Int. Cl. A61F 5/02; A61B 5/00; A61N 1/04; A61H 1/00

(52) U.S. Cl. CPC A61F 5/024; A61B 5/0053; A61H 2201/165; A61N 1/0484; A61H 1/00; A61B 5/0057

(57) ABSTRACT

A wearable orthotic appliance to which at least one corrective element for correcting neuromuscular imbalances may be removably attached is made of fabric assembly held in compressive tension when worn by a user, the fabric assembly including a cavity or chamber into which the corrective element may be placed and secured at any of a plurality of continuously-adjustable positions. Maintenance of the corrective element position during use of the appliance may be assisted by establishing a compression gradient over selected areas of the appliance during assembly. The wearable orthotic appliance may be a shirt, vest, body suit, or other garment or article of clothing, and the corrective elements may be weights, electrodes, or other discrete elements whose therapeutic effect depends on proper positioning relative to the wearer's torso or other body parts.

### BACKGROUND OF THE INVENTION

### 1. Field of the Invention

The invention relates to a wearable orthotic appliance to which corrective elements for correcting neuromuscular imbalances may be removably attached at therapeutically effective locations. The wearable orthotic appliance may be a shirt, vest, body suit, or other garment or article of clothing, and the corrective elements may be weights, electrodes, vibration-causing transducers, or other discrete elements whose therapeutic effect depends on proper positioning relative to the wearer's torso or other body parts.

The invention also relates to a wearable orthotic appliance in which corrective elements have been positioned by a process involving a clinician applying stimuli to an individual, observing individual reaction to the stimuli, positioning at least one weight in a cavity provided in the appliance, again applying the stimuli to the individual and observing the individual's reaction, and adjusting a position of the at least one weight within the cavity.

### 2. Description of Related Art

U.S. Pat. Nos. 7,708,673 and 7,156,792, both assigned to Motion Therapeutics, Inc. and incorporated herein by reference, describe orthotic appliances in the form of garments that are worn on the upper body and that include pockets or slits into which weights may be inserted. The resulting weighted garments are used to improve imbalance, instability, and/or rotational asymmetry, including but not limited to loss of balance in sitting or standing and difficulties in locomotion caused by neurological disorders such as multiple sclerosis, stroke, traumatic brain injury, Parkinson's disease, injuries, and congenital conditions. The weighted garments can also be used to address balance, stability, and symmetry issues not caused by injury or disorder, in order to improve performance in athletics or daily activities.

The present invention relates generally to orthotic appliances of the type disclosed in U.S. Pat. Nos. 7,708,673 and 7,156,792, but which have an improved construction that provides easier and more accurate, continuously adjustable positioning of the weights or other corrective elements. The improved construction includes replacement of weight-holding pockets with panels that enable a therapist or user to continuously adjust the positions of one or more corrective elements, using differential compressive tension to maintain positioning when the appliance is worn without the need for additional clinches or straps. The result is a lightweight, comfortable, orthotic appliance with enhanced ease-of-use and improved therapeutic effects.

By way of background, further description of the manner in which garment weighting or electrical stimulation achieves therapeutic effects, as well as weight placement protocols, and prior orthotic garment constructions are found on Motion Therapeutics' website ([http://www].motiontherapeutics.com) as well as in patents and publications by Cynthia Gibson-Horn, PT, the inventor of Motion-Therapeutics' BalanceWear® Balance-Based Torso-Weighting® (BBTW®) weighted garment technology describe on the Motion Therapeutics website. Further background including descriptions of other uses for weighted garments, biomechanical suits, and the like, for both orthotics and athletics, may be found, by way of example, in U.S. Pat. Nos. 4,268,917; 4,658,442; 4,602,387; 5,799,328; 5,810,699; 5,957,873.

An example of a garment that combines compression and weights is found in U.S. Pat. No. 8,944,974. This patent, however, does not provide for continuous adjustment of weight positions, but rather utilizes pockets to hold individual weights at discrete positions. Furthermore, the patent describes a unitary construction that is incapable of providing differential compression. While differential compression is provided in some embodiments, the differential compression is achieved by clinches.

An example of a garment that uses differential compressive tension to provide proprioceptive feedback to a wearer during movement is found in U.S. Pat. No. 9,445,932. The differential compressive tension is applied by tensor bands that are stitched into the garment, the material of the tensor bands being stiffer than the material used to make the remainder of the garment. However, U.S. Pat. No. 9,445,932 does not apply the differential compression to help secure corrective elements, and there is no disclosure of achieving the compressive tension during manufacture by subjecting panels to different tensile loads as they are stitched together, rather than by using material of different stiffness. As a result, the proprioceptive feedback provided by the garment disclosed in U.S. Pat. No. 9,445,932 cannot be adjusted by a clinician or user to compensate for specific individual neuromuscular deficiencies. Also, in the garment of U.S. Pat. No. 9,445,932, a compression gradient can only be established between the bands and surrounding material, rather than continuously across and within different panels of the same material.

As explained in the above-cited Motion Therapeutics patents, placement of the corrective elements is an iterative process that involves initial perturbation testing, placement of the corrective elements, further perturbation testing to evaluate the effects of the corrective element placement, adjustment of the corrective element positions, and repetition of the testing and adjustment steps until an acceptable therapeutic result is achieved. The tests may, for example, involve observing the reaction of an individual to applied linear and torsional forces in various directions while the individual is attempting to maintain a standing position, in order to determine how stable the individual is, or how quickly the individual responds to the applied linear and torsional forces. As each stage of testing is completed, the clinician must position weights or other corrective elements within the garment so as to correct for the imbalances exposed by the testing. By providing for continuous positioning of the corrective elements, i.e., positioning of the corrective elements at any arbitrary location within therapeutically significant areas of the garments, rather than requiring placement in discrete pockets, the clinician can more accurately position the corrective elements for better therapeutic effect.

While continuous positioning is desirable, the continuous positioning must still be achievable in a clinical setting and maintainable during use of the garment outside the clinical setting, and moreover provision must be made to allow the individual to recreate the original positioning when the corrective elements are removed from garment for laundering. None of the current pocket-based or orthotic appliances or compression garments is capable of enabling continuous positioning of corrective elements in a clinical setting, maintaining the positioning during use of the appliance outside the clinical setting, and yet of providing sufficient accessibility to enable a user to mark and thereby recreate the original positioning when the corrective elements are removed from the appliance outside the clinical setting (it should be appreciated that the term “clinical setting” in this context refers to any setting in which the positions of the corrective elements are established for therapeutic, athletic training, or other purposes).

### SUMMARY OF THE INVENTION

A wearable orthotic appliance to which at least one corrective element for correcting neuromuscular imbalances may be removably attached is made of fabric assembly held in compressive tension when worn by a user, the fabric assembly including a cavity or chamber into which the corrective element may be placed and secured at any of a plurality of continuously-adjustable positions.

Maintenance of the corrective element position during use of the appliance is preferably assisted by establishing a compression gradient over selected areas of the appliance during assembly.

The wearable orthotic appliance may be a shirt, vest, body suit, girdle, belt, or other garment or article of clothing, and the corrective elements may be weights, electrodes, vibration-causing transducers, or other discrete elements whose therapeutic effect depends on proper positioning relative to the wearer's torso or other body parts. The appliance itself is preferably made of a lightweight synthetic fabric material having elastic memory properties, such as Lycra®, Spandex®, a Lycra®-Spandex® blend, Neoprene®, or the like.

The orthotic appliance of the preferred embodiments is made up of a plurality of fabric panels, at least one of which includes a plurality of layers that form a corrective element positioning cavity or chamber, the corrective element positioning cavity or chamber including a material layer, coating, or other structure to which the corrective element may be removably secured, for example a layer to which an elastic Velcro® hook and loop fastener material may be removable adhered. The attachment layer is secured to at least one of an inner layer that defines one side of the cavity or chamber and an outer layer that define the second side of the cavity or chamber, the outer layer preferably forming an exterior layer of the panel. The attachment layer, inner layer, and outer layer are preferably assembled together to form a panel, which is then assembled to at least one other panel or fabric piece made of a flexible material to form the appliance.

It will be appreciated that, although a particular of arrangement of “layers” is described herein, layers may be combined or added while still achieving a desired compressive tension effect in both the panels that receive the corrective elements and the non-corrective-element receiving panels to which they are assembled, and further that the term “layers” may encompass both single and multiple ply fabric layers.

During assembly, the panels are preferably assembled together under tension. Although assembly under tension is not necessary to achieve a compression effect, the addition of tension by differential stretching of the panels as they are assembled together, enables a compression gradient to be achieved without the need to vary the stiffness of the panels using different materials. By increasing the tension in areas where the corrective elements are to be attached, compression is increased in those areas to assist in maintaining the position of the corrective elements when the orthotic appliance is worn by a user during ordinary and athletic activities. Differential compression may also be used to ensure a proper fit and to enhance the comfort of the user.

The use of a panel construction also enables ventilation panels to be included in the appliance, in order to provide increased heat dissipation. The ventilation panels may, for example, be made of a mesh material and provided at the sides of the appliance, adjacent the wearer's armpits or any other area of the torso not subject to stimulation by the corrective elements.

In the embodiment where the orthotic appliance is an upper body garment such as a shirt or vest, the garment may be tailored to have an added length to allow the free end at a lower side of the garment to be doubled over, increasing elastic retention of the weights.

In a preferred embodiment of the invention, the garment includes three cavities or chambers arranged to receive weights at positions most likely to provide therapeutic or performance benefits. The first is situated at the front of the garment and opening downwardly to cover the abdominal musculature, and the second and third cavities or chambers being situated on the back of the garment, one opening at the neck and the other at the bottom.

The corrective elements may be elastomeric weights embedded with metal, although other types of weights may be substituted without departing from the scope of the invention. The weights may be integrated with electrodes for causing electrical stimuli or vibration-causing transducers, the electrodes or vibration-causing transducers may be placed elsewhere on the garment, or the electrodes may be omitted.

In the case of embodiments in which the corrective elements are weights, the relatively distributed weight displacement of the embodiment enable electrodes or vibration-causing transducers to be freely located at positions outside the cavities or chambers.

The corrective elements are added to the orthotic appliance during an iterative process in which, following initial visual observation or perturbation testing, at least one corrective element is placed within a cavity or chamber of the appliance, further perturbation testing is carried out to evaluate the effects of the corrective element placement, the position of the corrective element within the cavity or chamber is adjusted without having to remove the corrective element entirely from the cavity or chamber and place it in a different cavity or chamber, and the testing and adjustment steps are repeated until an acceptable therapeutic result is achieved. The tests may, for example, involve observing the reaction of an individual to applied linear and torsional forces in various directions while the individual is attempting to maintain a sitting or standing position, in order to determine how stable the individual is, or how quickly the individual responds to the applied forces. The tests may also be applied to individuals in a seated position.

### BRIEF DESCRIPTION OF THE DRAWINGS

FIG. 1 is front view of a weighted garment constructed in accordance with the principles of a preferred embodiment of the invention.

FIG. 2 is a back view of the weighted garment of FIG. 1.

FIG. 3 is a cross-sectional view of the weighted garment of FIG. 1.

### DETAILED DESCRIPTION OF THE PREFERRED EMBODIMENTS

Throughout the following description and drawings, like reference numbers/characters refer to like elements. It should be understood that, although specific exemplary embodiments are discussed herein there is no intent to limit the scope of present invention to such embodiments. To the contrary, it should be understood that the exemplary embodiments discussed herein are for illustrative purposes, and that modified and alternative embodiments may be implemented without departing from the scope of the present invention.

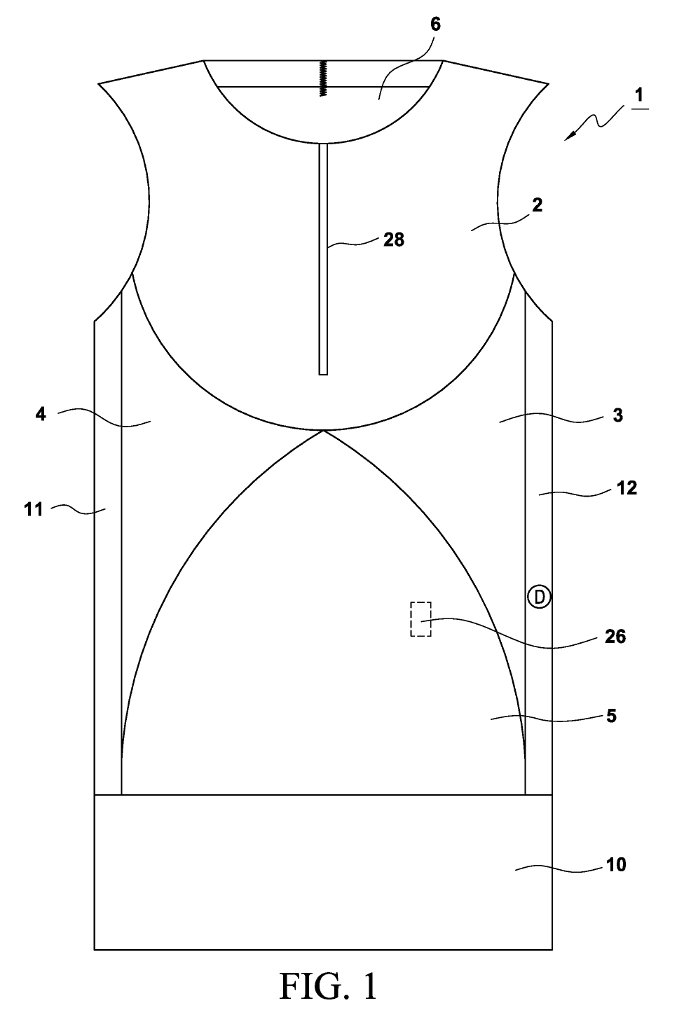

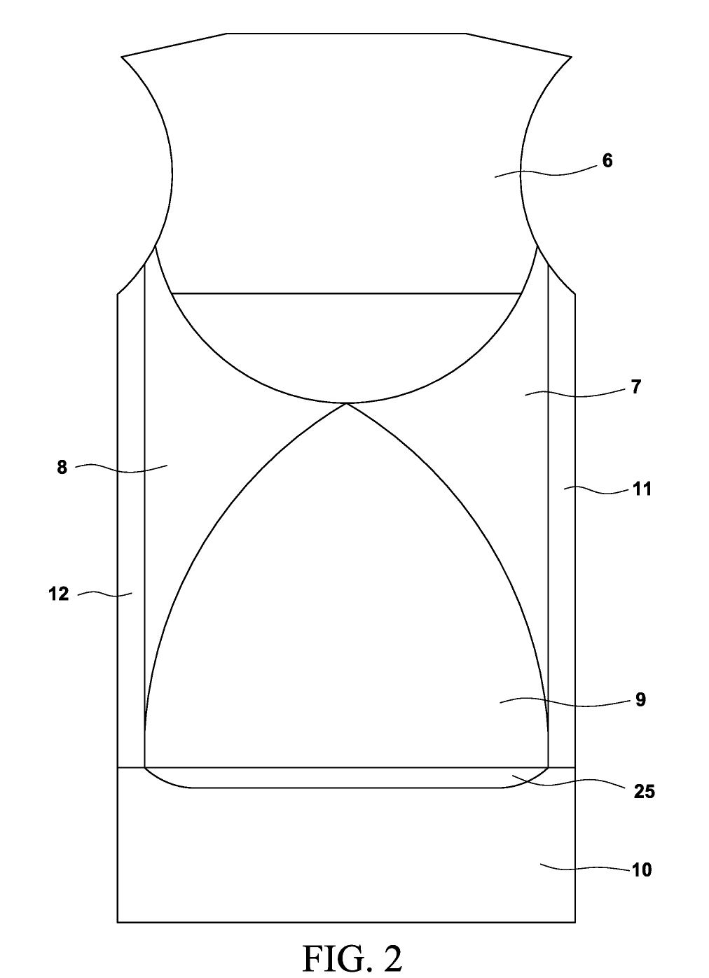

Although the present invention applies generally to an orthotic appliance, a preferred embodiment of the invention is in the form of a garment such as a vest or shirt. As illustrated in FIGS. 1 and 2, the weighted garment 1 constructed in accordance with the principles of a preferred embodiment of the invention is made of a light weight, breathable elastomeric material. The material may be a Lycra®, Spandex®, or Lycra®-Spandex® material, or another lightweight material having elastic memory properties, such as Neoprene®. When assembled into a garment, the garment is intended to fit snugly against the user's torso.

According to the preferred embodiment, the garment is constructed to exert a compressive force against the user's torso when the garment is worn. The compressive effect may be uniform or differential, i.e., the compressive force may vary at different locations across the garment. For example, a differential compressive effect may be used to apply a greater compression in areas where the corrective elements are to be positioned, and a lower compression in other areas. A differential compressive effect may be the result of different panel shapes, be achieved by assembling panels of the garment while under tension, whether uniform or varying, or by combinations of panel shape effects and application of uniform or varying tension.

As shown in FIGS. 1 and 2, the front and back sides of the garment are constructed of a plurality of solid panels 2-10, mesh panels 11 and 12, a foldable lower panel 13 that extends around both the front and back of the garment, each made of a resilient breathable material as described above, and an optional back crease panel 14 for added stability. The various panels are attached to each other at seams 12-20 having suitable resilience and strength to hold the panels together when under tension, for example by flat lock seam stitches or any other stitches or other material joining arrangements of techniques capable of joining panels of flexible material.

In the illustrated embodiment, three of the panels 8-10 are made up of multiple layers to form cavities or chambers into which corrective elements in the form of weights 25, electrodes (not shown), vibration-causing transducers (not shown), or other corrective elements may be fixed or removably inserted at varying positions within the chambers of cavities. It is preferred that the areas within the cavities or chambers to which the corrective elements are attached be large enough to cover an entire range of positions over which a corrective element may be adjusted after initial positioning, and that a panel be provided for each therapeutically significant area of the individual's torso. As a result, the cavities or chambers have a width that is a multiple (for example, 1.5, 2, 2.5, or 3) of the corresponding dimension of one of the corrective elements and extends over an entire region of the user's or individual's torso. In the illustrated embodiment, these regions include an area at the upper back, an area at the lower back, and an area in the front of the torso, with each area extending to both the right and left of the individual's vertical center or spine, although it is also possible to provide separate cavities or chambers on each side of the spine.

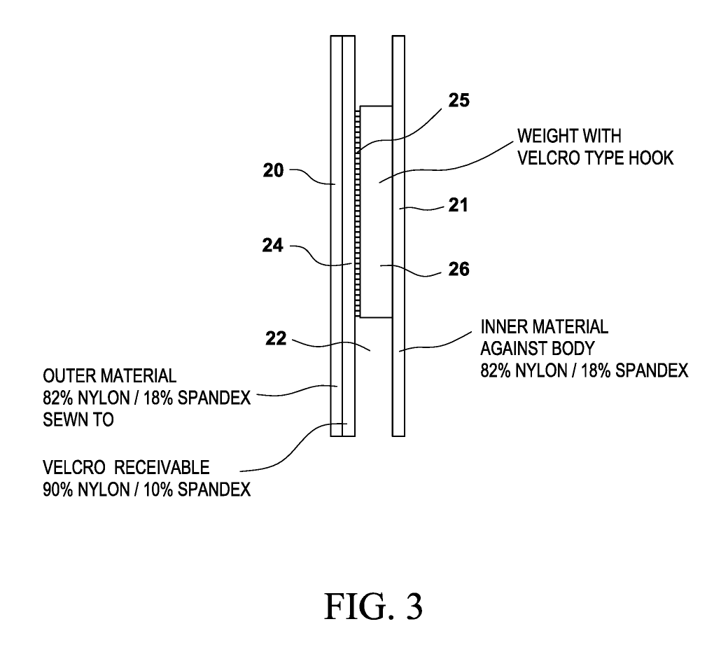

As shown in FIG. 3, the cavities or chambers may, for example, be formed of two 82% nylon/18% Spandex® power knit layers 20,21 that are attached to the interior side of the respective panels, with the cavity or chamber 22 being formed between the nylon/Spandex® layers 20 and 21, and a Velcro® attracting or fixing layer 24 made up of 90% nylon/10% Spandex® added to an interior side of one of the nylon/Spandex® layers, for example nylon/Spandex® layer 20, as illustrated in FIG. 3. The Velcro® attracting layer 24 attracts a corresponding Velcro® layer 25 on the weight 26 when the weight is positioned in the cavity or chamber 22. The three layers 20, 21, and 24 may be secured to each other and to the outer panels by any suitable method, including conventional stitching on three sides of each piece with the fourth side left open, and may further include closures or flaps for the openings. The lower back and front corrective element receiving panels 8 and 9 may, in the illustrated example, be further secured by folding of the bottom panel 10 over the lower openings, which also has the effect of increasing compression over the individual's lower torso.

Although a three-layer construction is shown, the invention is not limited to three layers or to the illustrated materials. For example, either the outer layer or the inner layer may itself be capable of holding the corrective elements, by including adhesive, magnetic, or mechanical fixing properties, and therefore the separate layer 24 may be omitted. Alternatively, additional layers may be added for any purpose, including structural reinforcement, perspiration removal, anti-microbial effects, or sensing of corrective element positions.

The shapes of the panels help determine the compressive force distribution across the garment and also the areas to which the corrective elements may be affixed. In the illustrated embodiment, corrective element receiving panels 8 and 9 have a generally triangular shape that extends approximately midway up the garment and is widest at the base of triangle. The panel 6 at the upper rear of the garment has a substantially rectangular shape, with upper and side curvatures to accommodate arm and neck shapes, and a slot for facilitating fitting of the garment over the user's head, and a closure such as a zipper 28. while the solid panel 2 at the upper front has a shape that accommodates a user's chest, arms, and neck. Hyperbolically triangular panels 3, 4, 7 and 8, rectangular side panels 11 and 12, and the continuous belt-like panel 10 complete the front and back sides of the garment. Those skilled in the art will appreciate that these shapes are described by way of example only, and not intended to limit the overall scope of the invention.

The corrective elements to be placed in the cavities or chambers may be conventional weights, such as parallelepiped-shaped elastomeric members into which metal has been embedded and that are available in units of 1/16, ⅛, ¼ or ½ pound, although it will be appreciated that the weights, dimensions, shapes, and composition of the weights may be varied without departing from the scope of the invention. Suitable electrode pads may include integrated power sources and be activated wirelessly, or connected by wires to a central controller and/or power source.

A process by which the corrective elements may be placed in the garment is to assemble a weighted orthotic appliance is generally described in U.S. Pat. Nos. 7,156,792 and 7,708,673. In general, the process is an iterative process that involves initial perturbation testing, placement of the corrective elements, further perturbation testing to evaluate the effects of the corrective element placement, adjustment of the corrective element positions, and repetition of the testing and adjustment steps until an acceptable therapeutic result is achieved. The testing steps involve observing the reaction of an individual to linear and torsional forces applied by the clinician in the form of pushes in various directions while the individual is attempting to maintain a standing position, in order to test the individual's stability or how quickly the individual responds to the applied forces. As each stage of testing is completed, the clinician positions one or more weights or other corrective elements within the cavities or chambers. By providing for continuous positioning of the corrective elements, i.e., positioning of the corrective elements at any arbitrary location within the cavities or chambers, rather than requiring placement in discrete pockets having a limited extent, the clinician can more easily and accurately adjust the position of the corrective elements for better therapeutic effect, without the need to remove the corrective elements from the cavities or chambers. Furthermore, by making the cavities or chambers more easily accessible, a user of the appliance can mark positions of the corrective elements within the cavities or chambers so as to more easily reposition the corrective elements after removal for laundering or dry cleaning.

It will be appreciated by those skilled in the art that, although the above-described process and appliance made by the process is very useful for the treatment of neuromuscular disorders, the invention is not limited to garments having corrective elements placed using the specific procedure described above. Instead, the differential compressive tension of the preferred embodiment assists in maintaining corrective element positioning for corrective elements that have been placed by any procedure, while the cavities or chambers providing in selected panels of the garment facilitate positioning of the corrective elements at desired locations on either side of the spine by any position-determining procedure, whether involving stimulus application and observation of individual reactions by a clinician, followed by retesting and adjustment, or any other manual or automated test, evaluation, and position-determining or calculating methods.

The side panels 11 and 12 may be attached to the solid fabric panels 1-10 by the same flat lock stitched seams used to hold attach the solid fabric panels to each other, or by any other fabric panel attachment means. The material of the mesh panels may be the same as the other panels, but the mesh panels are arranged to facilitate heat dissipation by increased ventilation. Although provided in the form of side panels positioned under the user's arms, the mesh panels 11 and 12 may alternatively, or in addition, be provided at other positions on the front or back of the garment, or may be omitted replaced by cut outs or inserts in the other non-corrective element attachment panels.

Finally, the illustrated embodiment may include a back crease to allow relative movement between the upper and lower back panels to be optionally provided for extra stability.

What is claimed is:

1. A wearable orthotic appliance to which at least one corrective element for correcting neuromuscular imbalances may be removably attached, comprising:
an assembly made up of resilient fabric held in compressive tension against a user's torso when worn, the assembly including at least one cavity or chamber into which the at least one corrective element may be placed and secured at any of a plurality of continuously-adjustable positions.

2. A wearable orthotic appliance as claimed in claim 1, wherein said compressive tension includes a compression gradient that assists in maintaining a position of the at least one corrective element within the cavity or chamber.

3. A wearable orthotic appliance as claimed in claim 1, wherein the wearable orthotic appliance is a garment or article of clothing.

4. A wearable orthotic appliance as claimed in claim 3, wherein the garment or article of clothing is a shirt or vest.

5. A wearable orthotic appliance as claimed in claim 1, wherein the corrective elements are weights whose therapeutic effect depends on proper positioning relative to the torso.

6. A wearable orthotic appliance as claimed in claim 1, wherein the corrective elements include electrodes for delivering neuromuscular stimulation.

7. A wearable orthotic appliance as claimed in claim 1, wherein the corrective elements include weights and electrodes.

8. A wearable orthotic appliance as claimed in claim 1, wherein the assembly made up of fabric includes a plurality of fabric panels stitched together, at least one of the panels being constructed of a plurality of layers that form the at least one corrective element positioning cavity or chamber, the corrective element positioning cavity or chamber including a material adapted to attract and removably secure the at least one corrective element.

9. A wearable orthotic appliance as claimed in claim 8, wherein the plurality of layers include an outside fabric layer, an inside fabric layer, and a third layer including said material adapted to attract and removably secure the at least one corrective element.

10. A wearable orthotic appliance as claimed in claim 9, wherein the third layer is made up of a Velcro® hook and loop fastener attracting material.

11. A wearable orthotic appliance as claimed in claim 10, wherein the third layer is secured to the inner layer or the outer layer of the cavity of chamber, and the inner and outer layer are secured to each other to form a discrete panel for assembly to panels made up of a single fabric layer.

12. A wearable orthotic appliance as claimed in claim 8, wherein the panels are assembled together under tension to generate the compressive tension.

13. A wearable orthotic appliance as claimed in claim 12, wherein a compression gradient is introduced during manufacture by differential stretching of the panels as they are assembled together, the differential tension increasing the tension in areas where the corrective elements are to be attached to assist in maintaining the position of the corrective elements when the orthotic appliance is worn by a user.

14. A wearable orthotic appliance as claimed in claim 8, wherein the panels include at least one ventilation panel and made of a single layer resilient mesh material.

15. A wearable orthotic appliance as claimed in claim 1, wherein the resilient fabric is a lightweight synthetic fabric material having elastic memory properties,

16. A wearable orthotic appliance as claimed in claim 1, fabric that includes Lycra®, Spandex®, a Lycra®-Spandex®, such as Neoprene®.

17. A wearable orthotic appliance as claimed in claim 1, wherein a number of the cavities or chambers is at least three.

18. A wearable orthotic appliance as claimed in claim 1, wherein a number of the cavities or chambers is three, and the cavities or chambers include a first cavity or chamber situated at a front of the appliance and opening downwardly from near the user's solar plexus, and second and third cavities or chambers situated at the back of the appliance, one opening at the user's neck and the other at a lower back area of the user's torso.

19. A wearable orthotic appliance made up of resilient fabric held in compressive tension against a user's torso when worn and that includes at least one weight positioned in a cavity or chamber of the appliance, the at least one weight being assembled to the wearable orthotic appliance by an iterative process of:
carrying out initial perturbation testing following initial perturbation testing,
placing at least one corrective element within the cavity or chamber of the appliance,
carrying out further perturbation testing to evaluate effects of the corrective element placement,
adjusting the position of the corrective element within the cavity or chamber without having to remove the corrective element entirely from the cavity or chamber and place it in a different cavity or chamber, and
repeating the testing and adjustment steps are repeated until an acceptable therapeutic result is achieved.

20. A wearable orthotic appliance as claimed in claim 19, wherein the steps of carrying out initial and further perturbation testing includes steps of a clinician applying linear and torsional forces to an individual in various directions while the individual is attempting to maintain a standing position, in order to determine how stable the individual is, or how quickly the individual responds, to the applied forces.
